(19) United States
(12) Patent Application Publication
(10) Pub. No.: US 2003/0001023 A1
(43) Pub. Date: Jan. 2, 2003

(54) Apparatus for providing a renewable source of cedar aroma in a storage area

(76) Inventor: Gordon Simms, York, PA (US)

Correspondence Address: JAMES RAY & ASSOCIATES, 2640 Pitcairn Road, Monroeville, PA, 15146, US

(21) Appl. No.: 10/094,222

(22) Filed: Mar. 8, 2002

Related U.S. Application Data
(60) Provisional application No. 60/274,162, filed on Mar. 8, 2001.

(51) Int. Cl. A61L 9/04

(52) U.S. Cl. 239/34

(57) ABSTRACT

The present invention provides an apparatus for providing a renewable source of cedar aroma in a storage area. The apparatus consists of a plurality of thin cedar curls having a predetermined shape and having a predetermined surface area. The apparatus further includes a perforated bag for containing such plurality of thin cedar curls.

### CROSS-REFERENCE TO RELATED APPLICATION

The invention taught in this patent application is closely related to the invention taught in the following co-pending U.S. Provisional Patten Application Serial No. 60/274,162 and was filed on Mar. 08, 2001. All the teachings therein are incorporated into this application by reference thereto.

### FIELD OF THE INVENTION

The present invention relates to an apparatus for protecting clothing in a storage area from moths and other insects and, more particularly, the present invention pertains to a perforated or mesh bag containing specially cut aromatic red cedar to provide a specific aroma which may be renewed by periodic squeezing of the enclosing member.

### BACKGROUND OF THE INVENTION

Historically, American Red Cedar has been used in many forms by Native Americans and early settlers for years to protect clothing from moths and other insects. All the conventional cedar products have to be scratched or sanded to renew the cedar aroma when the aroma appears to have faded. There has been a need for a more simpler method of renewing the aroma of the cedar to ensure continued protection.

### SUMMARY OF THE INVENTION

Therefore, the present invention provides an apparatus for providing a renewable source of cedar aroma in a storage area. The apparatus consists of a plurality of thin cedar curls having a predetermined shape and having a predetermined surface area. The apparatus further includes a perforated bag for containing such plurality of thin cedar curls.

### OBJECTS OF THE INVENTION

It is a primary object of the present invention to provide an apparatus which can introduce a large cedar aroma producing surface.

Another object of the present invention is to provide an apparatus which has a perforated bag which can be hung in a storage area for containing such cedar aroma producing surface.

Yet, another object of the invention is to provide an apparatus which can introduce a large cedar aroma producing surface in which the bag need only be squeezed to renew the cedar aroma.

These and various other objects and advantages of the invention will become apparent after a full reading of the following detailed description, particularly, when read in conjunction with the attached drawings as described below and the appended claims.

### BRIEF DESCRIPTION OF THE DRAWINGS

FIG. 1 is front view of a typical cedar “curl” according to an embodiment of the invention.

FIG. 2 is an isometric drawing of a mesh bag filled with thin cedar curls according to the present invention.

### BRIEF DESCRIPTION OF THE PRESENTLY PREFERRED AND VARIOUS ALTERNATE EMBODIMENTS OF THE INVENTION

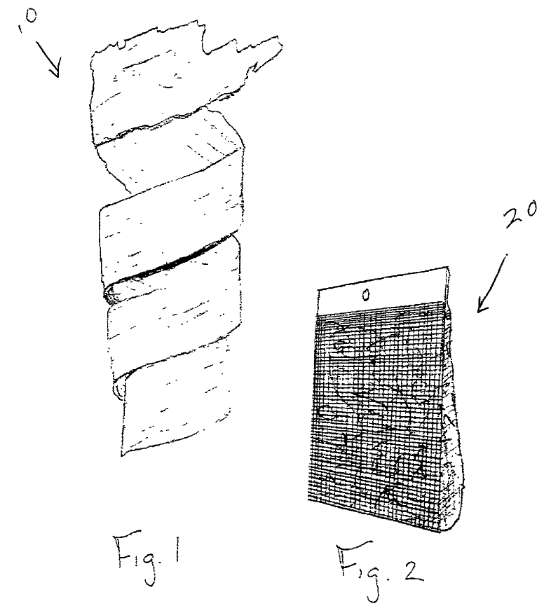

Reference is now made to FIGS. 1 and 2. Illustrated therein is a thin cedar curl, generally designated 10, typical of the geometry of the members which are stuffed into a mesh bag, generally designated 20. The bag 20 is referred to in this application as a mesh bag 20. However, the bag may not necessarily be specifically a mesh bag but is important that the bag be perforated so as to permit the cedar aroma to permeate into the storage area. In the presently preferred embodiment of the invention such bag 20 is a mesh bag.

The mesh bag 20 may be sized so as to provide a receptacle to enclose the required “cedar aroma producing area” for the storage area. This would mean that a small storage area could be protected with a relatively small bag which would contain only about 8 square feet of cedar producing exposed wood. While a larger bag containing up to about 175 square feet of exposed wood could be provided for a larger storage area.

The present invention provides an inexpensive, quick and convenient method of introducing a large cedar aroma producing surface in a relatively small perforated or mesh bag 20 which can be hung in a storage area (not shown). The unique geometry of the thin cedar curls 10 provides a spiral like curl stuffing which is easily squeezed so as to provide fresh cedar aroma and then returns to its original bulk to be squeezed again when there is a need for fresh aroma. The presently preferred spiral shape of the cedar coils 10 is easily seen in FIG. 1.

When the bag 20 is hung in a storage area the bag 20 is squeezed to generate the cedar aroma. As the aroma fades after a period of time the bag 20 containing the cedar curls 10 is squeezed again to renew the aroma. This process can be repeated again and again.

While both the presently preferred and a number of alternative embodiments of the present invention have been described in detail above it should be understood that various other adaptations and modifications of the present invention can be envisioned by those persons who are skilled in the relevant art without departing from either the spirit of the invention or the scope of the appended claims.

I claim:

1. An apparatus for providing a renewable source of cedar aroma in a storage area, said apparatus consisting of:
a. cedar material having a predetermined shape and having a predetermined surface area; and
b. a perforated container for containing said cedar material.

2. An apparatus for providing a renewable source of cedar aroma in a storage area, according to claim 1, wherein said cedar material is a curl.

3. An apparatus for providing a renewable source of cedar aroma in a storage area, according to claim 2, wherein said curl is a thin strip.

4. An apparatus for providing a renewable source of cedar aroma in a storage area, according to claim 3, wherein said apparatus includes a plurality of cedar curls.

5. An apparatus for providing a renewable source of cedar aroma in a storage area, according to claim 3, wherein said predetermined shape of said cedar curls is spiral.

6. An apparatus for providing a renewable source of cedar aroma in a storage area, according to claim 1, wherein said perforated container is selected from at least one of paper, cloth plastic, and wire.

7. An apparatus for providing a renewable source of cedar aroma in a storage area, according to claim 6, wherein said perforated container is a cloth mesh bag.

8. An apparatus for providing a renewable source of cedar aroma in a storage area, according to claim 1, wherein a size of said perforated container is varied depending on a size of such storage area.

9. An apparatus for providing a renewable source of cedar aroma in a storage area, according to claim 1, wherein said predetermined surface area of said cedar curls is between about 8 square feet and about 175 square feet.

10. An apparatus for providing a renewable source of cedar aroma in a storage area, according to claim 9, wherein said predetermined surface area of between about 8 and about 20 square feet of said cedar curls is for use in a small storage area.

11. An apparatus for providing a renewable source of cedar aroma in a storage area, according to claim 8, wherein said predetermined surface area of between about 75 and about 175 square feet of said cedar curls is for use in a large storage area.
